(19) United States
(12) Patent Application Publication
(10) Pub. No.: US 2024/0131286 A1
(43) Pub. Date: Apr. 25, 2024

(54) MEDICO-SURGICAL TUBES

(71) Applicant: SMITHS MEDICAL INTERNATIONAL LIMITED, Ashford (GB)

(72) Inventors: Laura Beth Morton, Sevenoaks (GB); Ayesha Bint-E-Siddiq, Folkestone (GB); Neil Steven Veasey, Ashford (GB); Christopher John Woosnam, London (GB); Andrew Thomas Jeffrey, Romney Marsh (GB)

(73) Assignee: SMITHS MEDICAL INTERNATIONAL LIMITED, Ashford (GB)

(21) Appl. No.: 18/277,527

(22) PCT Filed: Feb. 21, 2022
(86) PCT No.: PCT/GB2022/000025

§ 371 Date: Aug. 15, 2023

(30) Foreign Application Priority Data
Mar. 15, 2021 (GB) 2103603.3

(51) Int. Cl. A61M 16/04

(52) U.S. Cl. CPC A61M 16/0465; A61M 16/0434; A61M 2207/00

(57) ABSTRACT

A cuffed tracheal tube has a shaft (10) with an inner component (30) having an inflation lumen (23) extending along a rib (33) along the outside of the component. The rib (33) terminates a short distance rearwardly of the patient end (14) of the inner component. The inflation lumen (23) is closed by an outer layer (51) applied over the inner component (30) and covering the patient end of the rib. Two closely spaced openings (54) through the rib (33) are spaced in the inflatable region of a silicone sealing cuff (13) secured around the patient end of the shaft.

This invention relates to medico-surgical tubes of the kind including a shaft, an inflatable cuff towards the forward, patient end of the shaft and a lumen by which inflation fluid is supplied to or from the inside of the cuff.

Tracheal tubes are used to enable ventilation, respiration, or spontaneous breathing of a patient. Tracheostomy tubes are inserted into the trachea via a surgically formed opening in the neck so that one end locates in the trachea and the other end locates outside the patient adjacent the neck surface. Tracheostomy tubes can be inserted by different techniques, such as the surgical cut-down procedure carried out in an operating theatre, or a cricothyroidotomy procedure, which may be carried out in emergency situations.

Tracheostomy tubes are generally used for more long-term ventilation or where it is not possible to insert an airway through the mouth or nose. The patient is often conscious while breathing through a tracheostomy tube, which may be open to atmosphere or connected by tubing to some form of ventilator. The tube is secured in position by means of a flange fixed with the tube and positioned to extend outwardly on opposite sides of the tube.

Tracheostomy tubes can be made of various materials and are usually of a bendable plastics material such as PVC, polyurethane, or silicone. Tracheostomy tubes, and other medico-surgical tubes, are often also provided with some form of sealing means towards its patient end in order to form a seal with the trachea so that gas is confined to flow along the bore of the tube. Usually this sealing means takes the form of an inflatable sealing cuff inflated via an inflation lumen extending along the tube. The inflation lumen could be extruded or moulded with the wall of the shaft of the tube. In this case, the lumen must be opened externally in the region of the sealing cuff and closed distally of this at the patient end of the shaft to prevent the inflation fluid escaping. Usually, the lumen is opened in the region of the cuff by cutting through the outside of the shaft into the lumen. The lumen is closed at the patient end by a secondary operation, such as by inserting a plug of a material similar to that of the shaft into the open end of the lumen with an adhesive to bond it in place. This process can result in unacceptable rough edges around the occlusion, which can require the tube to be scrapped. This, and the time taken for the sealing operation, can lead to added costs.

It is an object of the present invention to provide an alternative medico-surgical tube.

According to one aspect of the present invention there is provided a medico-surgical tube of the above-specified kind, characterised in that the lumen is formed along the wall of an inner tubular component and extends to a first location towards the patient end of the component, that the lumen is arranged to open externally at a second location rearwardly of the first location and positioned along the length of the cuff within an inflatable region of the cuff, and that the tube includes an outer tubular component extending along a major part at least of the inner component and over the first location such as to occlude the lumen at the first location.

The lumen preferably extends along a rib projecting externally of the inner component. The rib preferably terminates a short distance rearwardly of the patient end of the inner component. The tube may include a reinforcement member between the inner and outer tubular components the reinforcement member extending a part way around the inner component leaving a longitudinal gap along which the rib extends. The lumen may open into the cuff at two places spaced longitudinally of one another. The cuff is preferably of a silicone. The tube may be a tracheal tube.

According to another aspect of the present invention there is provided a method of manufacture of a cuffed medico-surgical tube including the steps of forming an inner tubular component having a central bore and a longitudinal rib along the outside of the inner component and having an inflation lumen extending along the length of the rib and opening towards the patient end of the inner component, applying an outer layer over the inner component along a major part at least of its length and over the open patient end of the lumen so as to block an end of the lumen, forming an opening into the inflation lumen rearwardly of the blocked end, and applying a sealing cuff over the opening.

The method may include a step before applying the outer layer of removing a short length of the rib at its patient end so that the inflation lumen opens a short distance rearwardly of the patient end of the inner component. The method may include a step before applying the outer layer of applying a reinforcement member to the inner component to extend a part way around the inner component leaving a longitudinal gap along which the rib extends.

According to a further aspect of the present invention there is provided a tube made by a method according to the above other aspect of the present invention.

A tracheostomy tube according to the present invention will now be described, by way of example, with reference to the accompanying drawings in which:

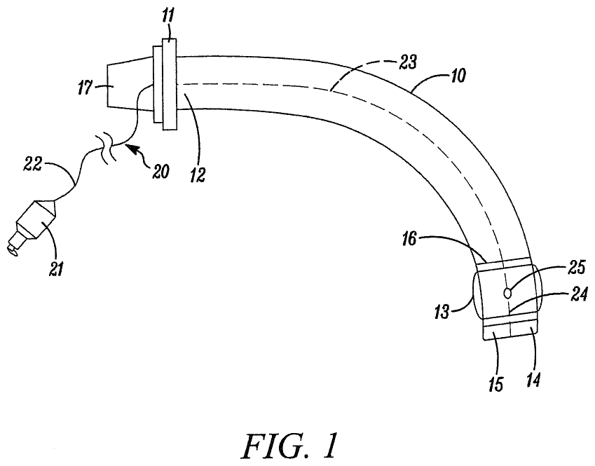

FIG. 1 is a side elevation view of the tracheostomy tube;

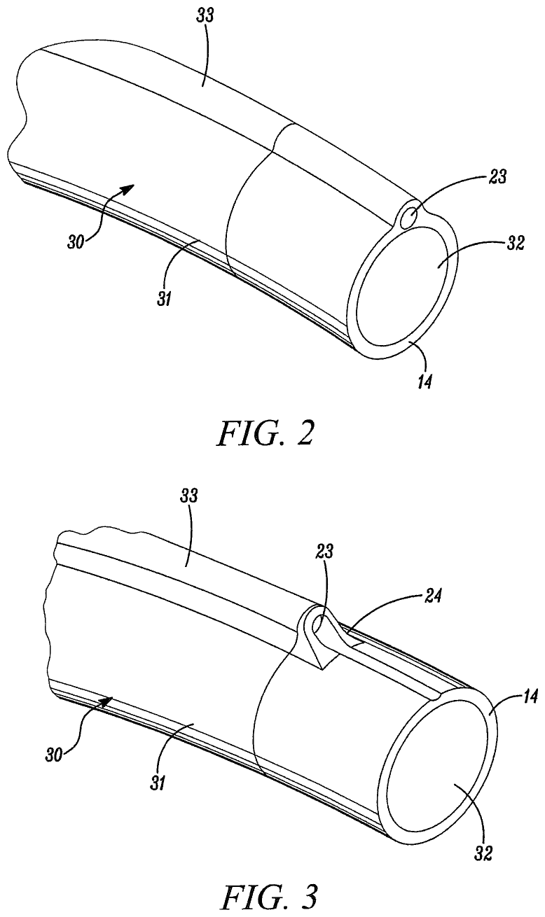

FIG. 2 is a perspective view of the forward, patient end of an inner component of the tube at a first stage of manufacture;

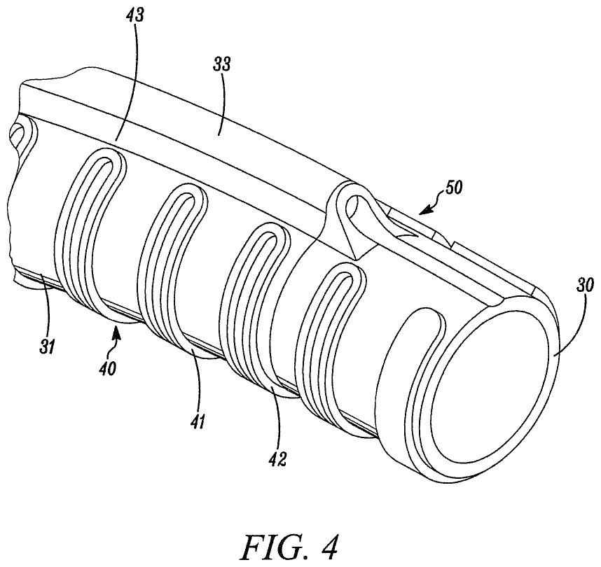

FIG. 3 is a perspective view of the component shown in FIG. 2 at a subsequent stage of manufacture;

FIG. 4 is a perspective view of the component shown in FIG. 3 with the addition of an outer reinforcement member; and

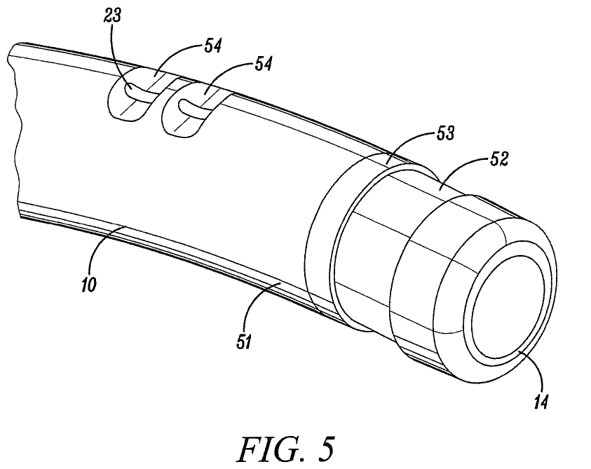

FIG. 5 is a perspective view of the tube shaft at a later stage of manufacture and before the addition of the sealing cuff.

With reference to FIG. 1. the tracheostomy tube 1 has a generally curved shaft 10, a laterally extending mounting or neck flange 11 attached with the shaft towards its rear, machine end 12, and a sealing cuff 13 attached with the shaft towards its distal, forward, or patient end 14 by attachment end collars 15 and 16. The cuff 13 is preferably of an elastic material, such as silicone, shaped to closely embrace the shaft 10 when deflated. The shaft 10 is also preferably of silicone although other materials could be used both for the cuff and the shaft. A male, tapered connector 17 is attached to the proximal, rear or machine end 12 of the shaft 10 for use in connecting the tube with a mating connector at one end of a breathing tube (not shown) extending to a ventilator or the like. Alternatively, the connector 17 can be left open where the patient is breathing spontaneously. The tube also includes means 20 for supplying an inflation fluid to and from the inside of the sealing cuff 13 for inflating and deflating the cuff. The inflation fluid used to inflate the cuff 13 could be a gas, such as air, or a liquid, such as water.

The means 20 for supplying an inflation fluid to the inside of the cuff 13 includes a combined inflation indicator and valved connector 21 connected to the machine end of inflation tubing 22. At its opposite, patient end, within the flange 11, the inflation tubing 22 connects with the machine end of an inflation lumen 23 formed within the wall of the shaft 10. The inflation lumen 23 is blocked at a first location 24 spaced a short distance rearwardly of the patient end 14. The inflation lumen 23 opens into the sealing cuff 13 at a second location 25 a short distance rearwardly of its blocked end 24 and between the two attachment collars 15 and 16, that is, within an inflatable region of the cuff.

Further details of the construction of the shaft 10 will become apparent from the following description of its method of manufacture with reference also to FIGS. 2 to 5.

The first step is to provide an inner tubular component 30 of the kind shown in FIG. 2. The tubular component 30 has a continuous cylindrical wall 31 with a central coaxial bore 32 of circular section. The component 30 also includes an integral longitudinal rib 33 extending along the entire length of its outside and having a circular bore parallel with the main bore 32 that provides the inflation lumen 23.

The next, second step, as shown in FIG. 3, is to remove, or skive, a short length of the rib 33 from the patient end 14 of the component 30 so that the inflation lumen 23 opens externally at the location 24 spaced a short distance from the patient end. This may be done by any conventional manual or automatic trimming operation such as involving machining or cutting. The length of the rib 33 removed is less than the distance between the patient end 14 and the location 25 where the inflation lumen 23 opens into the sealing cuff 13. The thickness of the rib 33 removed may include a small amount of the underlying wall 31, as shown, or may leave a small amount of the rib remaining on the wall.

At this stage, as shown in FIG. 4, it is possible to add an optimal reinforcement member 40 to the component 30 to form a sub-assembly 50. The reinforcement member 40 is in the form of a C-section coil 41 formed by a stiff element 42 extending part way around the component 30 in a zigzag or boustrophedon fashion leaving a longitudinal gap 43 in which the rib 33 extends. The reinforcement member 40 may be of a ferrous metal, a metal amalgam, a stiff polymer, ceramic, or the like. The reinforcement member 40 could be preformed to shape and then clipped onto, wrapped around, or slid along the tubular component 30. This reinforcement is optional and may be omitted, or alternative reinforcements could be used.

The next step, as shown in FIG. 5, is to place the sub-assembly 50 with any reinforcement 40, in a mould and to overmould an outer component 51 in the form of a compatible plastics or elastomer around the curved outer surface of the sub-assembly. Before this is carried out, a thin mandrel (not shown) is threaded along the inflation lumen 23 in the rib 33 from its machine end until its forward or patient end is close to, at, or just projecting from the open, patient end of the lumen. The purpose of the mandrel is to prevent material of the overmoulded layer 51 flowing rearwardly along the lumen 23 far enough to block the location at which the opening 25 into the sealing cuff 13 will be formed. After overmoulding, the mandrel is removed. The mould cavity may define external surface formations on the shaft 10 such as a shallow annular recess 52 and raised adhesive dam 53 to receive the collars 15 and 16 at the opposite ends of the cuff. Only the surface formations for the forward end of the cuff 13 are shown in FIG. 5. The overmoulding process, therefore, fully defines the tip at the patient end 14 of the shaft 10 without the need for any additional operation to occlude the end of the inflation lumen 23. After overmoulding the outer layer 51, one or more openings 54 are cut through the outer layer and the underlying rib 33 into the inflation lumen 23 so that this opens externally, centrally between the locations at which the sealing cuff 13 is attached by its collars 15 and 16, that is, in the inflatable region of the cuff. As shown in this example, the opening 25 into the cuff 13 is provided by two slots 54 cut through the rib 33 into the lumen 23 at places closely spaced from one another longitudinally along the shaft 10. Providing more than one outlet from the lumen 23 can help ensure a more even deflation of the sealing cuff 13 by reducing the risk that the cuff deflates with a portion contacting the shaft adjacent the inflation opening and trapping a volume of the inflation fluid within the cuff. It may also help promote a more even inflation of the cuff 13.

Connection can be made to the rear, machine end of the inflation lumen 23 by any conventional way. Typically, an opening is formed through the outside of the shaft 10 into the inflation lumen 23 in the region of the flange 11. One end of a stiff PTFE coupling (not shown) is inserted in the opening formed into the inflation lumen 23 and adhesively bonded in place. The forward end of the inflation line 22 is adhesively bonded over the opposite end of the coupling. The flange 11 is then slid over this connection to protect and conceal it, and is then bonded in place.

It is not essential that a patient end portion of the inflation lumen rib 33 be removed before overmoulding since the inner component 30 could be left in the state shown in FIG. 2, with or without the addition of an external reinforcement, and then overmoulded with the outer layer so that this blocks the open end of the inflation lumen 23 and, at the same time, forms a short cylindrical tip at the patient end.

The invention is not confined to tracheostomy tubes but could be used in other tubes having a sealing cuff. The sealing cuff could be of the self-inflating kind containing a resilient material such as a foam that gives the cuff a natural inflated shape and that is sucked down to a flatter configuration for insertion and removal by applying a negative pressure to the inflation lumen.

1-11. (canceled)

12. A medico-surgical tube including a shaft, an inflatable cuff towards the forward, patient end of the shaft and a lumen by which inflation fluid is supplied to or from the inside of the cuff, characterised in that the lumen is formed along the wall of an inner tubular component and extends to a first location towards the patient end of the component, that the lumen is arranged to open externally at a second location rearwardly of the first location and positioned along the length of the cuff within an inflatable region of the cuff, and that the tube includes an outer tubular component extending along a major part at least of the inner component and over the first location such as to occlude the lumen at the first location.

13. The medico-surgical tube according to claim 12, characterised in that the lumen extends along a rib projecting externally of the inner component.

14. The medico-surgical tube according to claim 13, characterised in that the rib terminates a short distance rearwardly of the patient end of the inner component.

15. The medico-surgical tube according to claim 13, characterised in that the tube includes a reinforcement member between the inner and outer tubular components, and that reinforcement member extends a part way around the inner component leaving a longitudinal gap along which the rib extends.

16. The medico-surgical tube according to claim 12, characterised in that the lumen opens into the cuff at two places spaced longitudinally of one another.

17. The medico-surgical tube according to claim 12, characterised in that the cuff is of a silicone.

18. The medico-surgical tube according to claim 12, characterised in that the tube is a tracheal tube.

19. A method of manufacture of a cuffed medico-surgical tube including the steps of forming an inner tubular component having a central bore and a longitudinal rib along the outside of the inner component and having an inflation lumen extending along the length of the rib and opening towards the patient end of the inner component, applying an outer layer over the inner component along a major part at least of its length and over the open patient end of the lumen so as to block an end of the lumen, forming an opening into the inflation lumen rearwardly of the blocked end, and applying a sealing cuff over the opening.

20. The method according to claim 19, characterised in that the method includes a step before applying the outer layer of removing a short length of the rib at its patient end so that the inflation lumen opens a short distance rearwardly of the patient end of the inner component.

21. The method according to claim 19, characterised in that the method includes a step before applying the outer layer of applying a reinforcement member to the inner component to extend a part way around the inner component leaving a longitudinal gap along which the rib extends.

22. A medico-surgical tube formed by the steps of:
forming an inner tubular component having a central bore and a longitudinal rib along the outside of the inner component and having an inflation lumen extending along the length of the rib and opening towards the patient end of the inner component;
applying an outer layer over the inner component along a major part at least of its length and over the open patient end of the lumen so as to block an end of the lumen;
forming an opening into the inflation lumen rearwardly of the blocked end; and
applying a sealing cuff over the opening.
